(19) United States
(12) Patent Application Publication
(10) Pub. No.: US 2024/0075232 A1
(43) Pub. Date: Mar. 7, 2024

(54) Bite block device

(71) Applicants: Donald Russell Peterson, Saint Charles, IL (US); John W. Birk, West Hartford, CT (US); Simon Kudernatsch, Saint Charles, IL (US)

(72) Inventors: Donald Russell Peterson, Saint Charles, IL (US); John W. Birk, West Hartford, CT (US); Simon Kudernatsch, Saint Charles, IL (US)

(21) Appl. No.: 17/929,379

(22) Filed: Sep. 2, 2022

(51) Int. Cl. A61M 16/04

(52) U.S. Cl. CPC A61M 16/0493; A61M 16/0495; A61M 2202/0208; A61M 2230/432

(57) ABSTRACT

A bite block to assist with upper Gastrointestinal (GI) endoscopy procedures. A main body of the bite block employs a rectangular shaped lumen to provide a large access port for insertion of variety of endoscopes and endoscopy equipment, individually or multiple instruments at the same time, and to guide them more precisely into the esophagus. The main body having a lumen shaped with respect to the anatomy of the oral cavity, where a curvature of the body is designed to act as a tongue depressor preventing airway blockage due to tongue collapse. Assigned channels run along the entire length of the device and are used to secure tubing in place for oxygen and monitor maximal concentration of carbon dioxide.

### FIELD OF THE INVENTION

The field of the invention is endoscopic medical procedures and, in particular, to a bite block for providing a working space during gastrointestinal endoscopic procedures.

### BACKGROUND OF THE INVENTION

A Gastrointestinal (GI) endoscopic procedure is a technique used by a doctor to facility placement of medical instruments such as tubes and scopes through the mouth of an individual to examine the person's digestive tract. An endoscope is a flexible tube with a light and camera attached to it wherein the doctor can examine the digestive track in real time using a display monitor, as well as capture clips of the video. An upper endoscopy is when an endoscope is passed through the mouth and throat and into the esophagus, allowing the doctor to view the esophagus, stomach, and upper part of the small intestine. An endoscopic ultrasound combines upper endoscopy and ultrasound examination to obtain images and information about various parts of the digestive tract.

Using an endoscope through the mouth opening for upper endoscopy procedures is commonly performed using a “bite block.” Bite blocks are found in a variety of sizes, designs, and models available with or without retaining straps to secure to the individual patient. Bite blocks protect delicate instruments and provides comfort for patients by acting as a mouth block during procedures. Bite blocks are typically made of a disposable material and may include 02 delivery.

Known bite blocks include: U.S. Pat. No. 5,174,284 discloses a bite block to protect against a patient's biting into an instrument inserted through the mouth in a medical procedure. As a patient bites on the block against a sloping channel, the block is seated in the mouth, and the tongue is firmly depressed.

U.S. Pat. No. 5,413,095 discloses a mouthpiece which directs oxygen into to the mouth of a patient, whether the oxygen is supplied to the patient by a single oxygen supply tube or by conventional nasal cannula extending from a common oxygen supply tube. In addition, the mouthpiece of the invention provides auxiliary openings through which fingers can be inserted into a patient's mouth so as to allow for manual manipulation of the mouthpiece once it is placed in the mouth.

U.S. Pat. No. 5,699,787 discloses an endotracheal system which contains a contoured mouthpiece capable of fitting between a patient's teeth. The endotracheal system also provides a medication injection line capable of delivering precise doses of medication directly to the patient's lungs. The medication may also be delivered in spray form when necessary. The endotracheal system includes a number of arresting elements, attached to the mouthpiece, which are capable of restricting the motion of the endotracheal tube. The endotracheal system may be secured to the patient by means of a flexible cord such as umbilical tape.

U.S. Pat. No. 7,946,288 discloses an device that contains a passage through which the patient can breathe by mouth. The device contains a port to which an oxygen line is connected and which delivers oxygen to the passage for inhalation by the patient. The device contains a sampling port which samples air in the passage and delivers sampled air to a monitoring device. The latter measures, for example, carbon dioxide content in the sampled gas, and issues a warning if carbon dioxide concentration is out-of-bounds. Phosphor material can be added to the device, to make the device glow in a darkened operating room, to allow technicians to easily find the patient's mouth and to insert tools, tubing, instruments and the like in the patient's mouth.

U.S. Pat. No. 9,186,473 discloses an endoscopic bite block, with teeth position defining regions which guide the subject's teeth to grip the bite block in their natural position, with the teeth of the upper jaw positioned further out than the lower jaw teeth. These teeth position defining regions are also preferably curved to match the curved shape of the jaw. The upper and lower parts of the front plate of the bite block may also be positioned at different distances out, so that upper and lower lip regions both contact the front plate snuggly. The bite block may be used with separate oral/nasal cannula, or may incorporate breath sampling or gas supply cannula. A flexible flapped curtain at the outer end of the bite block may be provided to largely enclose the inner volume of the bite block to enable more accurate capnographic sampling under conditions of wide-open mouth breathing.

U.S. Pat. No. 9,849,259 discloses a bite block comprising an insertion channel and an airway channel wherein the airway channel is adapted to receive exhaled breath and wherein the first insert channel is adapted to pass a medical instrument. There is provided herein, a bite block comprising an oral insert channel, wherein a portion of said channel opens into a cavity adapted to slidably receive an oral prong the oral insert channel is adapted to receive exhaled breath.

U.S. Pat. No. 10,300,234 discloses a bite block assembly adapted for capnography and oxygen delivery to a subject. The bite block assembly including a first capnography passageway adapted for passage therethrough of exhaled breath from the subject to a capnograph and a second oxygen delivery passageway, separate from the first passageway, adapted for passage therethrough of oxygen from an oxygen source to the mouth of the subject.

U.S. Pat. No. 10,653,307 discloses an oral airway device having a tubal body curved anteriorly and comprising a central lumen and one or more peripheral hollow passages for hosting a camera and/or one or more tools including a gastric suction tube, including oral airway devices in which the tubal body ends with a tongue and the wall of the tubal body encircles a central lumen. Oral airway devices also include those which comprise three channels, two of which are peripheral channels located in the flanks of the wall of the tubal body, the oral airway devices being compatible with a laryngoscope.

U.S. Pat. No. 10,821,248 discloses a courier airway device that includes a tubular structure that defines a central channel through which a medical diagnostic or therapeutic device can be inserted. The tubular structure also defines a ventilation duct for providing gases from a controlled ventilation machine to a patient. A cuff is attached to the central channel. Characteristically, the cuff is an inflatable balloon-like ring.

U.S. Patent Publication 2004/0129273 discloses a sedation and analgesia system having a bite block configured for integration and use with the system. The bite block facilitates access to the mouth while allowing the monitoring and delivery of gas via the oral and/or the nasal cavity where the monitoring and delivery are integrated with a sedation and analgesia system.

U.S. Patent Publication 2007/011344 discloses a bite block intended primarily for use with upper gastrointestinal endoscopy. The bite block comprises a unitary fabrication that fits into the mouth between the teeth or dental ridges and has a central passageway large enough to accommodate a gastroscope. The bite block also has a surface which lies exterior to the oral cavity and extends around the outer surface of the lips. The bite block also has a suction device which can be attached to a standard suction machine via flexible tubing or with an intermediary hollow wand. The suction device extends through the bite block into the intra-oral portion thereof where it forms an angle and allows suction drainage of pooled oral fluids from the cheek cavity. The interior tip of the suction device has multiple slit like openings circumferentially arranged to allow suction drainage of oral fluids without becoming occluded by the oral tissues.

U.S. Pat. No. 9,132,254 discloses various embodiments of mouthpieces for use during endoscopic procedures. According to various embodiments, a mouthpiece may include a shield, a primary instrument channel, a bite block, an oxygen administration channel and a tongue depressor. The shield, bite block, oxygen administration channel and tongue depressor may be integrally formed.

U.S. Pat. No. 4,495,945 discloses a bite block which is usefully employed in conjunction with endoscopy, bronchoscopy, endotracheal intubation and like medical-surgical procedures.

U.S. Pat. No. 6,257,238 discloses a bite block for upper gastrointestinal endoscopy having a tubular body with a proximal end and a distal end and defining a channel open at the proximal and distal ends, a shield connected to the tubular body at the proximal end, and a tongue depressor connected to the tubular body at the distal end. The tongue depressor comprises a plate connected to a bottom side of the distal end of the tubular body and is curved downwardly. The bite block has a chamber open to the channel in the bite block. A viscous bio-compatible lubricant in said chamber can contact and coat the end of an endoscope or other instrument when the instrument is inserted through the bite block. A removable tab can be pulled away from an opening into the chamber to expose the lubricant after the bite block has been placed in a patient's mouth. A lumen extends from the proximal end to the distal end of the tubular body, through the wall of the tubular body. An additional cannula or needle may be inserted through the lumen past the bite block and into the back of the throat, while isolating the additional needle from the endoscope.

U.S. Pat. No. 8,820,320 discloses a bite block providing an instrument lumen for use during transoral medical procedures may be made from a material having a Shore D hardness less than 60 for improved patient comfort. An air way lumen may be provided in side by side arrangement to the instrument lumen for maintaining the patient's airway. The air way may have a concave guiding channel for helping to guide the instruments down the esophagus.

U.S. Patent Publication 2007/025510 discloses a gastroscopic device for inspecting a body channel is described. The device comprises an insertion tube, an operating handle, a dispenser, which is detachably coupled to the insertion tube, a covering sleeve, which is retained within the dispenser, and a mouthpiece, which is provided with a means for arresting the dispenser within the mouthpiece. Upon arresting the dispenser within the mouthpiece and advancement of the insertion tube within the body channel, the covering sleeve extends from the dispenser and deploys around the insertion tube to protect from contamination that portion of the insertion tube, which had passed the dispenser.

What is needed in the art is improved bite block having a large enough guiding channel that can fit multiple surgical devices (e.g., endoscope, dilator, additional tubing, etc.) and, using the upper surface of the main body, direct them into the esophagus through the mouth and oropharynx. Additionally, side channels positioned on the outer lateral surfaces of a main body can accommodate standard size tubing for etCO2 monitoring and O2 supply without interfering with the surgical instruments inserted through the entrance. The unique shape of the main body functions as a guidance channel to the oral pharynx cavity and as a tongue depressor to maintain airway patency.

### SUMMARY OF THE INVENTION

Disclosed is a bite block to assist with upper Gastrointestinal (GI) endoscopy procedures. The bite block provides a safer access port for insertion of various surgical equipment (i.e. endoscope, dilator, tubing, etc.), to prevent airway blockage, and provide a means for intubation to supply oxygen (O2) to the patient and monitor maximal concentration of carbon dioxide (etCO2).

The device is inserted into the oral cavity and secured using an outer face attachment that fits around the individual's mouth with a latex-free rubber band. The main body employs a rectangular shaped lumen that is designed to provide a large enough access port for insertion of variety of endoscopes and endoscopy equipment, individually or multiple instruments at the same time, and to guide them more precisely and safely into the esophagus. The lumen of the main body is shaped with respect to the anatomy of the oral cavity, where the curvature is designed to act as a tongue depressor preventing airway blockage due to tongue collapse. A standard size tubing for etCO2 monitoring and O2 supply can be placed in side channels on both the outer lateral surfaces of the main body. The assigned channels run along the entire length of the device and secure the tubing in place safely.

An objective of the invention is to disclose a bite block having a lumen shaped with respect to the anatomy of the oral cavity, wherein a curvature provides a tongue depressor preventing airway blockage due to tongue collapse.

The primary significance of this device is that no device currently exists for upper GI endoscopy procedure that combines multiple functionalities and patient safety. The current devices that provide similar functionalities, such as tongue depressors and/or prevention of airway constrictions, are limited to only one or two of the features offered by this device. Current devices also do not provide an access channel to assist in the correct guidance of the endoscope in the mouth and oropharynx. Along with tongue depressor/guidance channel feature, this device allows for concurrent etCO2 monitoring and O2 delivery to the patient. Its unique shape and large lumen design provides an access channel along the entire length of the device body, which, when inserted, spans from the upper and lower lip to the base of the tongue. The lumen cross-section is large enough to fit multiple surgical tools at the same time and the strength of the device is intended to withstand significant amounts of biting force to prevent damage to the patient as well as to the instrumentation used during the procedures.

Still another objective of the invention is to disclose a bite block with channels to capture tubing wherein the correct length of the tubing can be determined prior to insertion of the device wherein the channels prevent tubing from slipping into the oropharynx.

Yet still another objective of the invention is to provide a one piece bite block suitable for bulk manufacturing via injection molding.

Yet still another objective of the invention is to provide a bite block that is low cost to manufacture, disposable or recyclable.

Still another objective of the invention is to provide a bite block with enough structural strength to withstand significant biting forces.

Still another objective of the invention is to provide a bite block having a unique rectangular main body shape that functions as a guidance channel to the oral pharynx cavity and as a tongue depressor to maintain airway patency. This function has recently become more significant as the rise in obesity rates has created more obstructive airway disease and provides for improved patient safety.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### DESCRIPTION OF THE DRAWINGS

FIG. 1 is a perspective view of the bite block;

FIG. 2 is a side view thereof.

FIG. 3 is a cross sectional end view taken along lines A-A of FIG. 2;

FIG. 4 is an enlarged detail view of section B of FIG. 3.

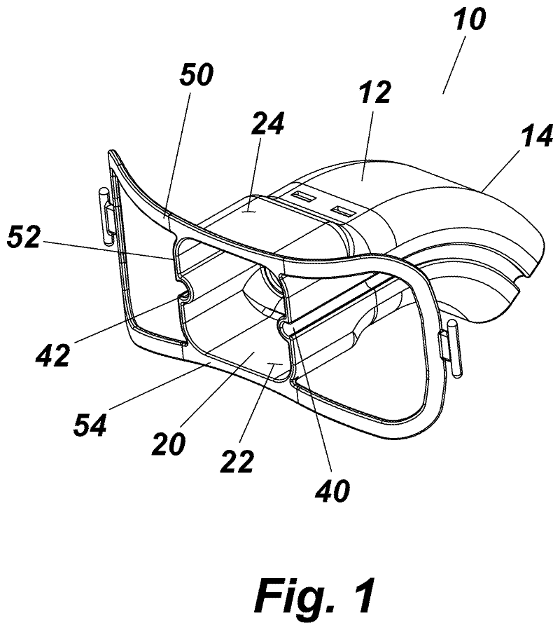

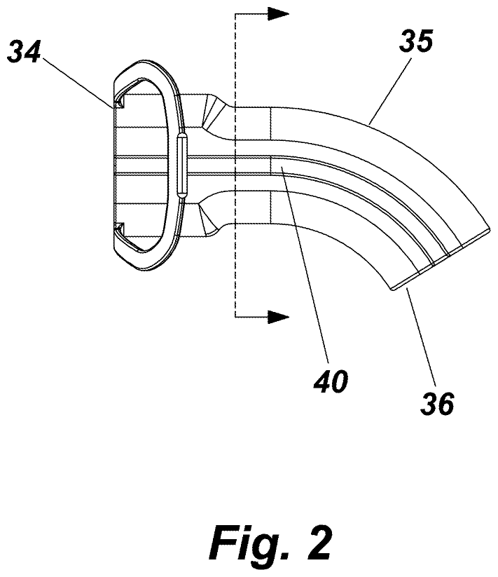

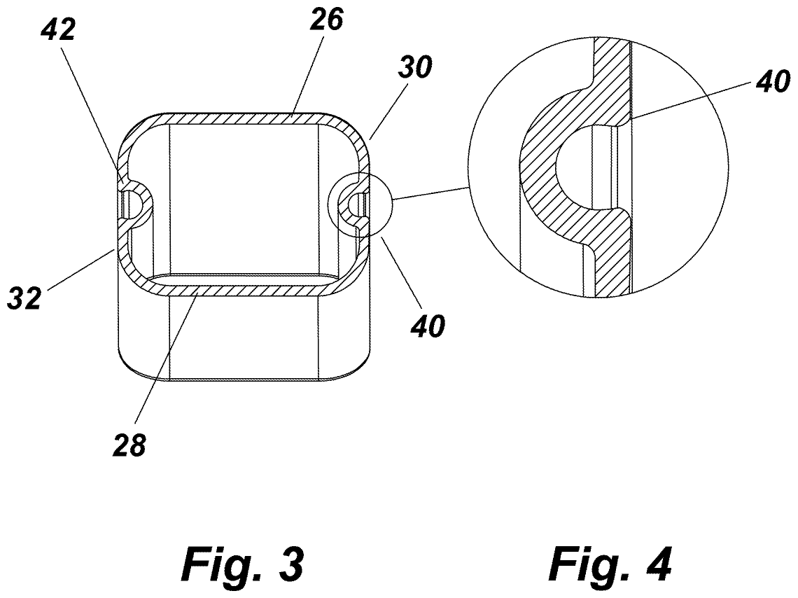

### DESCRIPTION OF THE PREFERRED EMBODIMENT

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

Referring to the figures, a bite block 10 is depicted to assist with upper Gastrointestinal (GI) endoscopy procedures. The bite block 10 comprises a tubular body member 12 having a continuous side wall 14. In the preferred embodiment, the body member 12 is constructed of a flexible material, the base 20 is rigid. The base 20 is defined by an inner surface 22 and an outer surface 24 forming a lumen opening 34. The continuous side wall 14 is further defined by an upper wall 26 spaced apart from a bottom wall 28 by first lateral side wall 30 and a second lateral side wall 32. The body member 12 has a curvature shape 35 to a trailing edge 36 positioned a predetermined distance from a leading edge 33. A first channel 40 formed integral with the first side wall 30 and extends from the leading edge 33 to the trailing edge 36. Similarly a second channel 42 is formed integral with the second side wall 32 extending from the leading edge 33 to the trailing edge 36.

An outer face attachment 50 having a rectangular shape is attached to an upper edge 52 of the leading edge 33 and the lower edge 54 of the leading edge 33. The outer face attachment 50 extends outward from the first side wall 30 and the second side wall 32.

The bite block device 10 is inserted into the oral cavity and secured using the outer face attachment 50 that fits around the individual's mouth with a latex-free rubber band, not shown. The main body 12 employs a rectangular shaped lumen that is designed to provide a large enough access port for insertion of variety of endoscopes and endoscopy equipment, individually or multiple instruments at the same time, and to guide them more precisely and safely into the esophagus. The base 20 with the main body 12 is shaped with respect to the anatomy of the oral cavity, where the curvature 35 is designed to act as a tongue depressor preventing airway blockage due to tongue collapse. A standard size tubing for etCO2 monitoring and O2 supply can be placed in side channels 40, 42 on the outer lateral surfaces of the main body. The assigned channels run along the entire length of the device from the leading edge 34 to the trailing edge 36 and secure the tubing, not shown, frictionally in place.

The bite block device 10 is intended to combine multiple functionalities including assistance with esophageal intubation, patient injury prevention and monitoring, and equipment protection. The outer face attachment 50 of the bite block device 10 is designed to comfortably fit around the mouth and is to be additionally secured around the head using a latex free rubber band. The entrance is designed to keep the mouth open and to provide an access port for insertion of variety of surgical instruments. The body 12 of the device is optimized in shape to guide the instruments into the esophagus, where the curved shape 35 at the distal end of the device functions as a tongue depressor by securing the base of the tongue in place. In order to prevent damage to teeth and gums as well as to surgical instruments, the device has enough strength to withstand significant amounts of biting force. In order to monitor patient oxygenation levels and to supply oxygen, the device also has side channels on both the lateral surfaces of the main body for easy placement of standard sized tubing compliant with etCO2 monitoring and O2 delivery equipment commonly used in the operation room (OR). The side channels are designed for the press-fit of tubing (etCO2 and O2), the channels keep the tubing in place throughout the procedure. Placing the tubing on the outer surface of the device body prevents any interference with the surgical equipment used during the procedure in the lumen. The channel allows the medical provider to set a proper length of tubing prior to device insertion into the oral cavity. The channel preventing the tubing from slipping further into the oropharynx.

The device is intended for large batch manufacturing via injection molding using FDA-approved, biocompatible, low-density polymer materials with high enough structural strength. To minimize the number of pieces with potential to separate and enter patient's body, the entire device is to be manufactured as a complete unit.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more elements, possesses those one or more elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

All patents and publications mentioned in this specification are indicative of the levels of those skilled in the art to which the invention pertains. It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A bite block device comprising:
a base having a continuous side wall forming a rectangular shaped lumen defined by an inner surface and an outer surface, said continuous side wall further defined by an upper wall spaced apart from a bottom wall by a first and second lateral side walls;
a flexible body member having a continuous side wall defined by an inner surface and an outer surface, said continuous side wall further defined by an upper wall spaced apart from a bottom wall by a first and second lateral side walls, said body member have a leading edge releasably secured to said base forming a lumen with a curvature shape;
a first channel formed integral with said first side wall extending from said leading edge to said trailing edge of said base and body member;
a second channel formed integral with said second side wall extending from said leading edge to said trailing edge of said base and body member; and
an outer face attachment having a rectangular shape attached to an upper edge of said leading edge and said lower edge of said leading edge, said outer face attachment extending outward from said first and second side walls;
wherein said body member having a curvature shape is constructed and arranged to follow the anatomy of an individual's oral cavity and provide tongue depression.

2. The bite block device accordingly to claim 1 wherein each said channel is constructed and arranged to hold tubing.

3. The bite block device accordingly to claim 2 wherein said tubing is for delivery of oxygen.

4. The bite block device accordingly to claim 2 wherein said tubing is for monitoring etCO2.

5. The bite block device accordingly to claim 1 wherein said outer face attachment is constructed and arranged to be secured in position by a latex free rubber band.

6. The bite block device accordingly to claim 1 wherein said curvature shape is approximated by a lower edge of said leading edge in a plane substantially equal to an upper edge of said trailing edge.

7. The bite block device accordingly to claim 1 wherein said base is constructed from a single piece of injection molded FDA-approved biocompatible low-density polymer material.

8. The bite block device accordingly to claim 1 wherein said body member and face attachment are constructed from a single piece of injection molded FDA-approved biocompatible low-density polymer material having flexibility properties.
